(19) United States
(12) Patent Application Publication
(10) Pub. No.: US 2024/0130778 A1
(43) Pub. Date: Apr. 25, 2024

(54) SURGICAL INSTRUMENTS, SYSTEMS, AND METHODS INCORPORATING ULTRASONIC AND ELECTROSURGICAL FUNCTIONALITY

(71) Applicant: Covidien LP, Mansfield, MA (US)

(72) Inventors: Thomas E. Drochner, Longmont, CO (US); Matthew S. Cowley, Frederick, CO (US); Kenlyn S. Bonn, Lakewood, CO (US); James R. Fagan, Erie, CO (US); Michael B. Lyons, Boulder, CO (US); David J. Van Tol, Boulder, CO (US)

(73) Assignee: Covidien LP, Mansfield, MA (US)

(21) Appl. No.: 18/279,578

(22) PCT Filed: Mar. 1, 2022
(86) PCT No.: PCT/US2022/018318

§ 371 Date: Aug. 29, 2023

Related U.S. Application Data
(60) Provisional application No. 63/155,517, filed on Mar. 2, 2021.

(51) Int. Cl. A61B 18/14; A61B 18/00; A61B 18/12

(52) U.S. Cl. CPC A61B 18/1445; A61B 18/1206; A61B 2018/00083; A61B 2018/00607; A61B 2018/0063; A61B 2018/00994

(57) ABSTRACT

A surgical end effector includes an ultrasonic blade and a jaw member. The blade is adapted to receive ultrasonic and electrosurgical energy and defines a distal face. The jaw member is movable relative to the blade from a spaced-apart to an approximated position for clamping tissue and includes a structural body having a body portion and a distal cap portion. The distal cap portion defines an electrode that receives electrosurgical energy. The jaw member further includes a jaw liner engaged within the body portion and extending therefrom towards the blade such that, in the approximated position, the jaw liner contacts the blade to define a gap distance between the electrode and the distal face to facilitate bipolar electrosurgical tissue treatment upon conduction of bipolar energy between the electrode and the distal face and through tissue in contact therewith.

### CROSS-REFERENCE TO RELATED APPLICATIONS

This application is a 371 National Stage Application of International Application No. PCT/US2022/018318, filed Mar. 1, 2022, which claims the benefit of, and priority to, U.S. Provisional Patent Application No. 63/155,517, filed on Mar. 2, 2021, the entire contents of which are hereby incorporated herein by reference.

### FIELD

The present disclosure relates to energy-based surgical instruments and, more particularly, to surgical instruments, systems, and methods incorporating ultrasonic and electrosurgical functionality to facilitate energy-based tissue treatment.

### BACKGROUND

Ultrasonic surgical instruments and systems utilize ultrasonic energy, i.e., ultrasonic vibrations, to treat tissue. More specifically, ultrasonic surgical instruments and systems utilize mechanical vibration energy transmitted at ultrasonic frequencies to treat tissue. An ultrasonic surgical device may include, for example, an ultrasonic blade and a clamp mechanism to enable clamping of tissue against the blade. Ultrasonic energy transmitted to the blade causes the blade to vibrate at very high frequencies, which allows for heating tissue to treat tissue clamped against or otherwise in contact with the blade.

Electrosurgical instruments and systems conduct Radio Frequency (RF) energy through tissue to treat tissue. An electrosurgical instrument or system may be configured to conduct bipolar RF energy between oppositely charged electrodes and through tissue, e.g., tissue clamped between the electrodes or otherwise in contact therewith, to treat tissue. Alternatively or additionally, an electrosurgical instrument or system may be configured to deliver monopolar RF energy from an active electrode to tissue in contact with the electrode, with the energy returning via a remote return electrode device to complete the circuit.

### SUMMARY

As used herein, the term “distal” refers to the portion that is described which is further from an operator (whether a human surgeon or a surgical robot), while the term “proximal” refers to the portion that is being described which is closer to the operator. Terms including “generally,” “about,” “substantially,” and the like, as utilized herein, are meant to encompass variations, e.g., manufacturing tolerances, material tolerances, use and environmental tolerances, measurement variations, and/or other variations, up to and including plus or minus 10 percent. Further, any or all of the aspects described herein, to the extent consistent, may be used in conjunction with any or all of the other aspects described herein.

Provided in accordance with aspects of the present disclosure is an end effector assembly of a surgical instrument. The end effector assembly includes an ultrasonic blade adapted to receive ultrasonic energy to vibrate the ultrasonic blade. The ultrasonic blade is also adapted to connect to a source of electrosurgical energy. The ultrasonic blade defines a distal face. The end effector assembly also includes a jaw member movable relative to the ultrasonic blade from a spaced-apart position to an approximated position for clamping tissue therebetween. The jaw member includes a structural body and a jaw liner. The structural body has a body portion and a distal cap portion. At least a portion of the distal cap portion of the structural body defines an electrode adapted to connect to the source of electrosurgical energy at a potential different from a potential of the ultrasonic blade. The jaw liner is engaged within the body portion of the structural body and extends therefrom towards the ultrasonic blade such that, in the approximated position, the jaw liner contacts the ultrasonic blade to define a gap distance between the electrode and the distal face of the ultrasonic blade. The gap distance facilitates bipolar electrosurgical tissue treatment upon conduction of bipolar energy between the electrode and the distal face of the ultrasonic blade and through tissue in contact therewith.

In an aspect of the present disclosure, the entire distal cap portion defines the electrode. Alternatively, a portion of the distal cap portion is electrically insulative and a distal cap electrode disposed on or within the electrically insulative portion defines the electrode.

In another aspect of the present disclosure, a distal-most extent of the electrode and a distal-most extent of the distal face of the ultrasonic blade are substantially aligned with one another in the approximated position.

In still another aspect of the present disclosure, the distal cap portion encloses a distal face of the jaw liner.

In yet another aspect of the present disclosure, the jaw liner is formed from an electrically-insulative material. Additionally or alternatively, the jaw liner is formed from a more-compliant material and the structural body is formed from a more-rigid material.

In still yet another aspect of the present disclosure, the body portion of the structural body includes first and second electrode surfaces extending along opposing sides of the jaw liner. The first and second electrode surfaces are adapted to connect to the source of electrosurgical energy at a potential different from the potential of the ultrasonic blade to enable conduction of bipolar energy between the first and second electrode surfaces and the ultrasonic blade and through tissue clamped therebetween in the approximated position.

In another aspect of the present disclosure, the electrode and the first and second electrode surfaces are independently energizable. Alternatively or additionally, the electrode and the first and second electrode surfaces are electrically coupled to one another.

In yet another aspect of the present disclosure, the gap distance is a first gap distance and, in the approximated position, the ultrasonic blade and the first and second electrode surfaces define a second gap distance therebetween that may be similar to or different from the first gap distance.

A method of surgery provided in accordance with aspects of the present disclosure includes clamping a jaw member against an ultrasonic blade such that a jaw liner of the jaw member contacts the ultrasonic blade to define a gap distance between a distal face of the ultrasonic blade and a distal cap portion of the jaw member. The jaw member includes the jaw liner and a structural body having a body portion and the distal cap portion. The jaw liner is engaged within the body portion of the structural body and the distal cap portion includes an electrode. The method further includes positioning the electrode and the distal face in contact with tissue and energizing the electrode to a first potential and the distal face to a second potential different from the first potential to conduct bipolar energy between the electrode and the distal face and through the tissue in contact therewith to treat the tissue.

In an aspect of the present disclosure, the method further includes collectively moving the jaw member and the ultrasonic blade into or along tissue to further treat tissue with bipolar energy.

### BRIEF DESCRIPTION OF THE DRAWINGS

The above and other aspects and features of the present disclosure will become more apparent in light of the following detailed description when taken in conjunction with the accompanying drawings wherein like reference numerals identify similar or identical elements.

FIG. 1 illustrates a surgical system provided in accordance with the present disclosure including a surgical instrument, a surgical generator and, in some aspects, a return electrode device;

FIG. 2 is a schematic illustration of a robotic surgical system provided in accordance with the present disclosure;

FIG. 3 is a longitudinal cross-sectional view of an end effector assembly of the surgical instrument of FIG. 1;

FIGS. 4 and 5 are transverse cross-sectional and distal end views, respectively, of the end effector assembly of the surgical instrument of FIG. 1;

FIG. 6 is a distal end view of another configuration of the end effector assembly of the surgical instrument of FIG. 1; and

FIGS. 7 and 8 are transverse cross-sectional and distal end views, respectively, of other configurations of the end effector assembly of the surgical instrument of FIG. 1.

### DETAILED DESCRIPTION

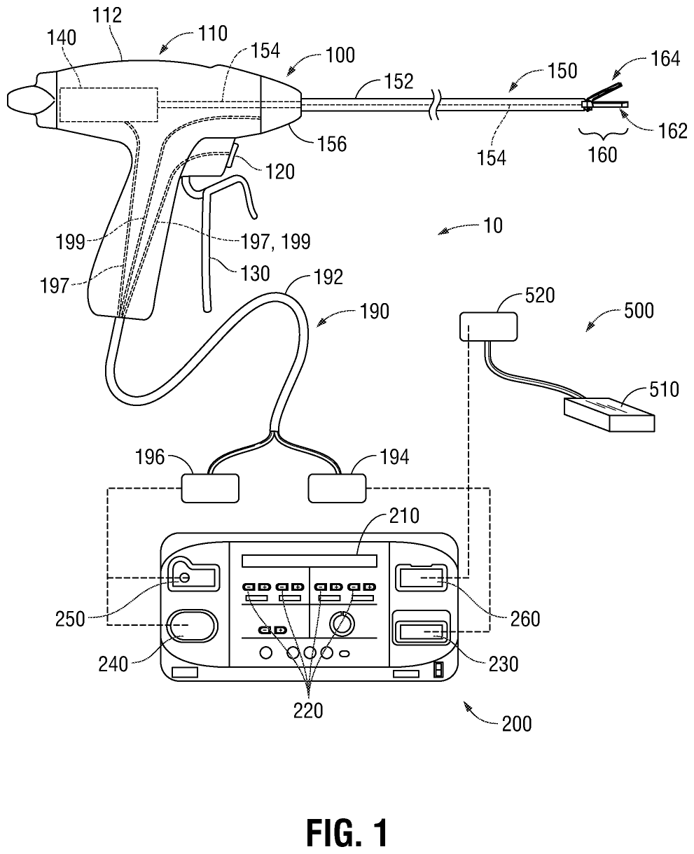

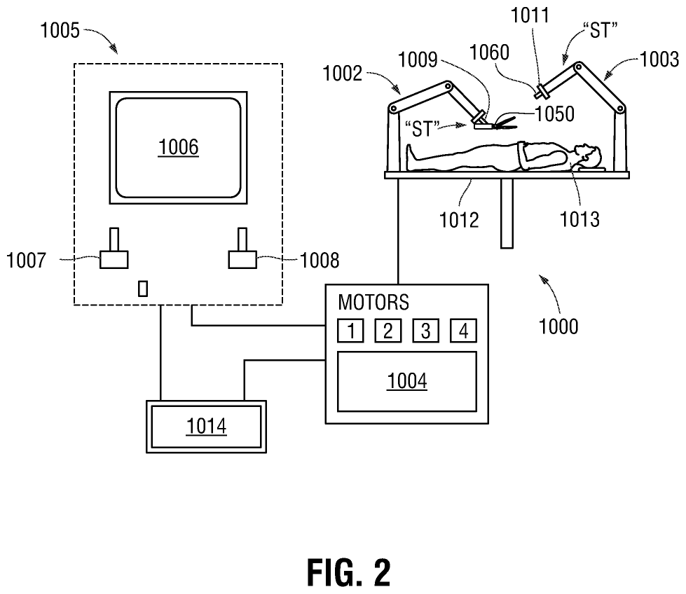

Referring to FIG. 1, a surgical system provided in accordance with aspects of the present disclosure is shown generally identified by reference numeral 10 including a surgical instrument 100, a surgical generator 200, and, in some aspects, a return electrode device 500, e.g., including a return pad 510. Surgical instrument 100 includes a handle assembly 110, an elongated assembly 150 extending distally from handle assembly 110, an end effector assembly 160 disposed at a distal end of elongated assembly 150, and a cable assembly 190 operably coupled with handle assembly 110 and extending therefrom for connection to surgical generator 200. As an alternative to handle assembly 110, surgical instrument 100 may include a robotic attachment housing for releasable engagement with a robotic arm of a robotic surgical system such as, for example, robotic surgical system 1000 (FIG. 2) detailed below.

Surgical generator 200 includes a display 210, a plurality user interface features 220, e.g., buttons, touch screens, switches, etc., an ultrasonic plug port 230, a bipolar electrosurgical plug port 240, and active and return monopolar electrosurgical plug ports 250, 260, respectively. Surgical generator 200 is configured to produce ultrasonic drive signals for output through ultrasonic plug port 230 to surgical instrument 100 to activate surgical instrument 100 in an ultrasonic mode and to provide electrosurgical energy, e.g., RF bipolar energy, for output through bipolar electrosurgical plug port 240 and/or RF monopolar energy for output through active monopolar electrosurgical port 250 to surgical instrument 100 to activate surgical instrument 100 in one or more electrosurgical modes. It is also contemplated that one or more common ports (not shown) may be configured to act as any two or more of ports 230-260. In monopolar configurations, plug 520 of return electrode device 500 is configured to connect to return monopolar electrosurgical plug port 260.

Continuing with reference to FIG. 1, handle assembly 110 includes a housing 112 defining a body portion and a fixed handle portion. Handle assembly 110 further includes an activation button 120 and a clamp trigger 130. The body portion of housing 112 is configured to support an ultrasonic transducer 140. Ultrasonic transducer 140 may be permanently engaged with the body portion of housing 112 or removable therefrom. Ultrasonic transducer 140 includes a piezoelectric stack or other suitable ultrasonic transducer components electrically coupled to surgical generator 200, e.g., via one or more of first electrical lead wires 197, to enable communication of ultrasonic drive signals to ultrasonic transducer 140 to drive ultrasonic transducer 140 to produce ultrasonic vibration energy that is transmitted along a waveguide 154 of elongated assembly 150 to blade 162 of end effector assembly 160 of elongated assembly 150, as detailed below. An activation button 120 is disposed on housing 112 and coupled to or between ultrasonic transducer 140 and/or surgical generator 200, e.g., via one or more of first electrical lead wires 197, to enable activation of ultrasonic transducer 140 in response to depression of activation button 120. In some configurations, activation button 120 may include an ON/OFF switch. In other configurations, activation button 120 may include multiple actuation switches to enable activation from an OFF position to different actuated positions corresponding to different activation settings, e.g., a first actuated position corresponding to a first activation setting and a second actuated position corresponding to a second activation setting. In still other configurations, separate activation buttons may be provided, e.g., a first actuation button for activating a first activation setting and a second activation button for activating a second activation setting.

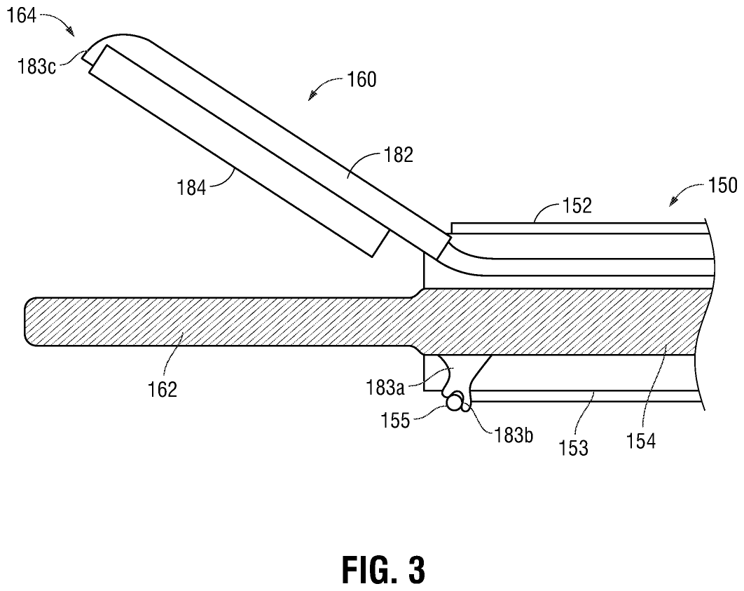

Elongated assembly 150 of surgical instrument 100 includes an outer drive sleeve 152, an inner support sleeve 153 (FIG. 3) disposed within outer drive sleeve 152, a waveguide 154 extending through inner support sleeve 153 (FIG. 3), a drive assembly (not shown), a rotation knob 156, and an end effector assembly 160 including a blade 162 and a jaw member 164. Rotation knob 156 is rotatable in either direction to rotate elongated assembly 150 in either direction relative to handle assembly 110. The drive assembly operably couples a proximal portion of outer drive sleeve 152 to clamp trigger 130 of handle assembly 110. A distal portion of outer drive sleeve 152 is operably coupled to jaw member 164 and a distal end of inner support sleeve 153 (FIG. 3) pivotably supports jaw member 164. As such, clamp trigger 130 is selectively actuatable to thereby move outer drive sleeve 152 about inner support sleeve 153 (FIG. 3) to pivot jaw member 164 relative to blade 162 of end effector assembly 160 from a spaced apart position to an approximated position for clamping tissue between jaw member 164 and blade 162. The configuration of outer and inner sleeves 152, 153 (FIG. 3) may be reversed, e.g., wherein outer sleeve 152 is the support sleeve and inner sleeve 153 (FIG. 3) is the drive sleeve. Other suitable drive structures as opposed to a sleeve are also contemplated such as, for example, drive rods, drive cables, drive screws, etc.

Referring still to FIG. 1, the drive assembly may be tuned to provide a jaw clamping force, or jaw clamping force within a jaw clamping force range, to tissue clamped between jaw member 164 and blade 162 or may include a force limiting feature whereby the clamping force applied to tissue clamped between jaw member 164 and blade 162 is limited to a particular jaw clamping force or a jaw clamping force within a jaw clamping force range.

Waveguide 154, as noted above, extends from handle assembly 110 through the inner support sleeve. Waveguide 154 includes blade 162 disposed at a distal end thereof. Blade 162 may be integrally formed with waveguide 154, separately formed and subsequently attached (permanently or removably) to waveguide 154, or otherwise operably coupled with waveguide 154. Waveguide 154 and/or blade 162 may be formed from titanium, a titanium alloy, or other suitable electrically conductive material(s), although non-conductive materials are also contemplated. Waveguide 154 includes a proximal connector (not shown), e.g., a threaded male connector, configured for engagement, e.g., threaded engagement within a threaded female receiver, of ultrasonic transducer 140 such that ultrasonic motion produced by ultrasonic transducer 140 is transmitted along waveguide 154 to blade 162 for treating tissue clamped between blade 162 and jaw member 164 or positioned adjacent to blade 162.

Cable assembly 190 of surgical instrument 100 includes a cable 192, an ultrasonic plug 194, and an electrosurgical plug 196. Ultrasonic plug 194 is configured for connection with ultrasonic plug port 230 of surgical generator 200 while electrosurgical plug 196 is configured for connection with bipolar electrosurgical plug port 240 of surgical generator 200 and/or active monopolar electrosurgical plug port 250 of surgical generator 200. In configurations where generator 200 includes a common port, cable assembly 190 may include a common plug (not shown) configured to act as both the ultrasonic plug 194 and the electrosurgical plug 196. Plural first electrical lead wires 197 electrically coupled to ultrasonic plug 194 extend through cable 192 and into handle assembly 110 for electrical connection to ultrasonic transducer 140 and/or activation button 120 to enable the selective supply of ultrasonic drive signals from surgical generator 200 to ultrasonic transducer 140 upon activation of activation button 120 in an ultrasonic mode. In addition, plural second electrical lead wires 199 are electrically coupled to electrosurgical plug 196 and extend through cable 192 into handle assembly 110. In bipolar configurations, separate second electrical lead wires 199 are electrically coupled to waveguide 154 and jaw member 164 (and/or different portions of jaw member 164) such that, as detailed below, bipolar electrosurgical energy may be conducted between blade 162 and jaw member 164 (and/or between different portions of jaw member 164). In monopolar configurations, an electrical lead wire 199 is electrically coupled to waveguide 154 such that, as also detailed below, monopolar electrosurgical energy may be supplied to tissue from blade 162. Alternatively, an electrical lead wire 199 may electrically couple to jaw member 164 in the monopolar configuration to enable monopolar electrosurgical energy to be supplied to tissue from jaw member 164. One or more second electrical lead wires 199 is electrically coupled to activation button 120 to enable the selective supply of electrosurgical energy from surgical generator 200 to waveguide 154 and/or jaw member 164 upon activation of activation button 120 in an electrosurgical mode.

As an alternative to a remote generator 200, surgical system 10 may be at least partially cordless in that it incorporates an ultrasonic generator, an electrosurgical generator, and/or a power source, e.g., a battery, thereon or therein. In this manner, the connections from surgical instrument 100 to external devices, e.g., generator(s) and/or power source(s), is reduced or eliminated.

With reference to FIG. 2, a robotic surgical system in accordance with the aspects and features of the present disclosure is shown generally identified by reference numeral 1000. For the purposes herein, robotic surgical system 1000 is generally described. Aspects and features of robotic surgical system 1000 not germane to the understanding of the present disclosure are omitted to avoid obscuring the aspects and features of the present disclosure in unnecessary detail.

Robotic surgical system 1000 generally includes a plurality of robot arms 1002, 1003; a control device 1004; and an operating console 1005 coupled with control device 1004. Operating console 1005 may include a display device 1006, which may be set up in particular to display three dimensional images; and manual input devices 1007, 1008, by means of which a person (not shown), for example a surgeon, may be able to telemanipulate robot arms 1002, 1003 in a first operating mode. Robotic surgical system 1000 may be configured for use on a patient 1013 lying on a patient table 1012 to be treated in a minimally invasive manner. Robotic surgical system 1000 may further include a database 1014, in particular coupled to control device 1004, in which are stored, for example, pre-operative data from patient 1013 and/or anatomical atlases.

Each of the robot arms 1002, 1003 may include a plurality of members, which are connected through joints, and an attaching device 1009, 1011, to which may be attached, for example, a surgical tool “ST” supporting an end effector 1050, 1060. One of the surgical tools “ST” may be ultrasonic surgical instrument 100 (FIG. 1), e.g., configured for use in both an ultrasonic mode and an electrosurgical (bipolar and/or monopolar) mode, wherein manual actuation features, e.g., actuation button 120 (FIG. 1), clamp lever 130 (FIG. 1), etc., are replaced with robotic inputs. In such configurations, robotic surgical system 1000 may include or be configured to connect to an ultrasonic generator, an electrosurgical generator, and/or a power source. The other surgical tool “ST” may include any other suitable surgical instrument, e.g., an endoscopic camera, other surgical tool, etc. Robot arms 1002, 1003 may be driven by electric drives, e.g., motors, that are connected to control device 1004. Control device 1004 (e.g., a computer) may be configured to activate the motors, in particular by means of a computer program, in such a way that robot arms 1002, 1003, their attaching devices 1009, 1011, and, thus, the surgical tools “ST” execute a desired movement and/or function according to a corresponding input from manual input devices 1007, 1008, respectively. Control device 1004 may also be configured in such a way that it regulates the movement of robot arms 1002, 1003 and/or of the motors.

Referring to FIG. 3, end effector assembly 160 of surgical instrument 100 of surgical system 10 (FIG. 1) is detailed, although end effector assembly 160 may be utilized with any other suitable surgical instrument and/or surgical system. End effector assembly 160 includes a blade 162 and a jaw member 164. Blade 162 may define a linear configuration, may define a curved configuration, or may define any other suitable configuration, e.g., straight and/or curved surfaces, portions, and/or sections; one or more convex and/or concave surfaces, portions, and/or sections; etc. With respect to curved configurations, blade 162, more specifically, may be curved in any direction relative to jaw member 164, for example, such that the distal tip of blade 162 is curved towards jaw member 164, away from jaw member 164, or laterally (in either direction) relative to jaw member 164. Further, blade 162 may be formed to include multiple curves in similar directions, multiple curves in different directions within a single plane, and/or multiple curves in different directions in different planes. In addition, blade 162 may additionally or alternatively be formed to include any suitable features, e.g., a tapered configuration, various different cross-sectional configurations along its length, cut outs, indents, edges, protrusions, straight surfaces, curved surfaces, angled surfaces, wide edges, narrow edges, and/or other features.

Blade 162 may define a polygonal, rounded polygonal, or any other suitable cross-sectional configuration(s) (see FIG. 5). Waveguide 154 or at least the portion of waveguide 154 proximally adjacent blade 162, may define a cylindrical shaped configuration. Plural tapered surfaces (not shown) may interconnect the cylindrically shaped waveguide 154 with the polygonal (rounded edge polygonal, or other suitable shape) configuration of blade 162 to define smooth transitions between the body of waveguide 154 and blade 162.

Blade 162 may be wholly or selectively coated with a suitable material, e.g., a non-stick material, an electrically insulative material, an electrically conductive material, combinations thereof, etc. Suitable coatings and/or methods of applying coatings include but are not limited to Teflon®, polyphenylene oxide (PPO), deposited liquid ceramic insulative coatings; thermally sprayed coatings, e.g., thermally sprayed ceramic; Plasma Electrolytic Oxidation (PEO) coatings; anodization coatings; sputtered coatings, e.g., silica; ElectroBond® coating available from Surface Solutions Group of Chicago, IL, USA; or other suitable coatings and/or methods of applying coatings.

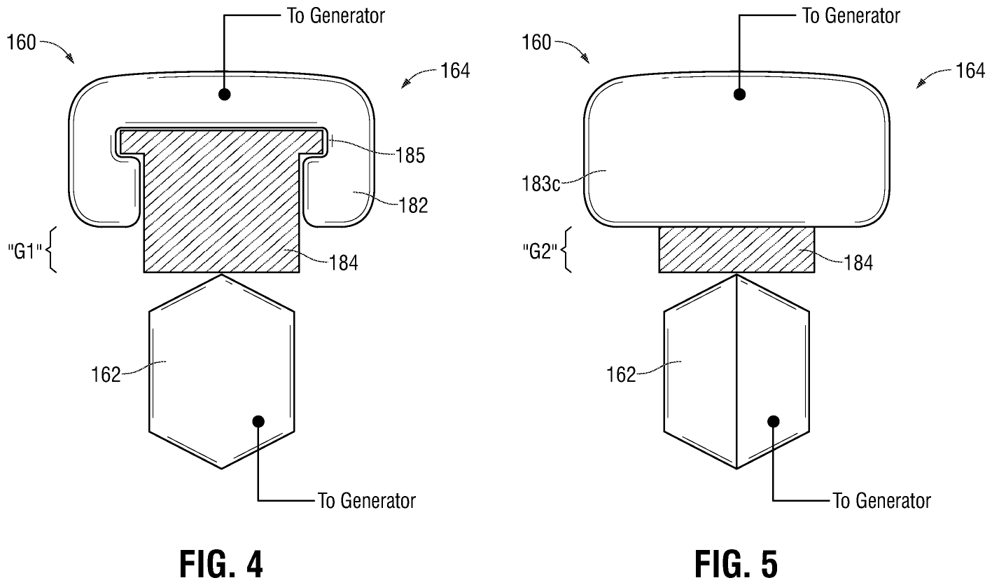

With additional reference to FIGS. 4 and 5, blade 162, as noted above, in addition to receiving ultrasonic energy transmitted along waveguide 154 from ultrasonic transducer 140 (FIG. 1), is adapted to connect to generator 200 (FIG. 1) to enable the supply of RF energy to blade 162 for conduction to tissue in contact therewith. In bipolar configurations, RF energy is conducted between blade 162 and jaw member 164 (or between portions of jaw member 164 and/or blade 162) and through tissue disposed therebetween to treat tissue. In monopolar configurations, RF energy is conducted from blade 162, serving as the active electrode, to tissue in contact therewith and is ultimately returned to generator 200 (FIG. 1) via return device 500 (FIG. 1), serving as the passive or return electrode.

Jaw member 164 of end effector assembly 160 includes a more rigid structural body 182 and a more compliant jaw liner 184. Structural body 182 may be formed from an electrically conductive material, e.g., stainless steel, and/or may include electrically conductive portions. Structural body 182 includes a pair of proximal flanges 183a that are pivotably coupled to the inner support sleeve 153 via receipt of pivot bosses (not shown) of proximal flanges 183a within corresponding openings (not shown) defined within the inner support sleeve 153 and operably coupled with outer drive sleeve 152 via a drive pin 155 secured relative to outer drive sleeve 152 and pivotably received within apertures 183b defined within proximal flanges 183a. As such, sliding of outer drive sleeve 152 about inner support sleeve 153 pivots jaw member 164 relative to blade 162 from a spaced apart position to an approximated position to clamp tissue between jaw liner 184 of jaw member 164 and blade 162.

Structural body 182, or a portion(s) thereof, may be adapted to connect to a source of electrosurgical energy, e.g., generator 200 (FIG. 1), and, in a bipolar electrosurgical mode, is charged to a different potential as compared to blade 162 to enable the conduction of bipolar electrosurgical (e.g., RF) energy through tissue clamped therebetween, to treat the tissue. In a monopolar electrosurgical mode, structural body 182 may be un-energized, may be charged to the same potential as compared to blade 162 (thus both defining the active electrode), or may be energized while blade 162 is not energized (wherein structural body 182 defines the active electrode). In either monopolar configuration, energy is returned to generator 200 (FIG. 1) via return device 500 (FIG. 1), which serves as the passive or return electrode.

Jaw liner 184 is shaped complementary to a cavity 185 (FIG. 4) defined within structural body 182, e.g., defining a T-shaped configuration, to facilitate receipt and retention therein, although other configurations are also contemplated. Jaw liner 184 is fabricated from an electrically insulative, compliant material such as, for example, polytetrafluoroethylene (PTFE). The compliance of jaw liner 184 enables blade 162 to vibrate while in contact with jaw liner 184 without damaging components of ultrasonic surgical instrument 100 (FIG. 1) and without compromising the hold on tissue clamped between jaw member 164 and blade 162. Jaw liner 184 extends from structural body 182 towards blade 162 to inhibit contact between structural body 182 and blade 162 in the approximated position of jaw member 164. The insulation of jaw liner 184 maintains electrical isolation between blade 162 and structural body 182 of jaw member 164, thereby inhibiting shorting.

Structural body 182 of jaw member 164 includes a distal cap portion 183c that extends distally beyond and encloses at least a portion of the distal face of jaw liner 184 (see FIG. 5). Blade 162 may extend to substantially the same distal extent as distal cap portion 183c when jaw member 164 is disposed in the approximated position. More specifically, in aspects, in the approximated position of jaw member 164, a distal-most extent of distal cap portion 183c may be substantially aligned with a distal-most extent of blade 162, e.g., both extending to a vertical plane perpendicular to a longitudinal axis of blade 162. Other configurations are also contemplated such as, for example, wherein different portions of distal cap portion 183c and blade 162 are aligned and/or wherein distal cap portion 183c and blade 162 extend distally different extents. The distal face of distal cap portion 183c (or a portion thereof) and/or the distal face of blade 162 (or a portion thereof) may define any suitable configuration similar or different from one another such as, for example, planar, spherical, ovoid, polyhedral, etc.

Although jaw liner 184, in aspects, does not extend to the distal-most extent of end effector assembly 160, jaw liner 184 still maintains a gap distance between structural body 182 and blade 162 and, more specifically, between distal cap portion 183c of structural body 182 and blade 162 in the approximated position of jaw member 164 due to jaw liner 184 extending further towards blade 162 as compared to structural body 182 so as to contact blade 162 in the approximated position. As can be appreciated, due to differences in structural body 182, jaw liner 184, and/or blade 162, the gap distance between distal cap portion 183c of structural body 182 and blade 162 (FIG. 5) need not be the same as the gap distance between blade 162 and the body portion of structural body 182 extending along opposed sides of jaw liner 184. Indeed, in aspects, the gap distances are different; in other aspects, the gap distances are the same. This gap distance(s) is described in greater detail below. As an alternative or in addition to jaw liner 184 defining the gap distance(s), other suitable stop structures or stop mechanisms, e.g., associated with proximal flanges 183a, elongated assembly 150, handle assembly 110, etc., may be provided.

Continuing with reference to FIGS. 4 and 5, as detailed above, structural body 182 may be adapted to connect to a source of electrosurgical energy, e.g., generator 200 (FIG. 1), and, in a bipolar electrosurgical mode, is charged to a different potential as compared to blade 162 to enable the conduction of bipolar electrosurgical (e.g., RF) energy through tissue clamped therebetween, to treat, e.g., seal, the tissue. In such a configuration, a gap distance “G1” may be defined between blade 162 and the body portion of structural body 182 extending along opposed sides of jaw liner 184 (see FIG. 4) in the approximated position (measured with jaw liner 184 in contact with blade 162 in the approximated position without tissue clamped therebetween), as shown in FIG. 4. Additionally or alternatively, the distal portion of end effector assembly 160 (acting as a probe with jaw member 164 in the approximated position without tissue therebetween) may be advanced distally into and/or moved transversely across tissue such that tissue (and/or a conductive medium such as saline) contacts and electrically connects the distal face of blade 162 and distal cap portion 183c of structural body 182 to enable the conduction of bipolar electrosurgical (e.g., RF) energy through the tissue to treat, e.g., spot coagulate, cut, or otherwise treat, the tissue (see FIG. 5). The gap distance “G2” defined between distal cap portion 183c of structural body 182 and blade 162 in such configurations may be defined when jaw liner 184 and blade 162 are in the approximated position in contact with one another, as shown in FIG. 5. In aspects, the gap distance “G1” is greater than the gap distance “G2.” In other aspects, the gap distances “G1” and “G2” are substantially similar; in other aspects, the gap distance “G2” is greater than the gap distance “G1.”

Controlling the gap distance between the electrodes, whether in a configuration where end effector assembly 160 clamps tissue to act as a tissue sealing device or in a configuration where end effector assembly 160 acts as a bipolar probe, facilitates effective tissue treatment. Depending upon the treatment to be performed and the corresponding use configuration (clamp device for tissue sealing versus probe spot coagulation, for example), end effector assembly 160 may be configured to provide a suitable gap distance “G1,” “G2” or gap distance within a suitable gap distance range.

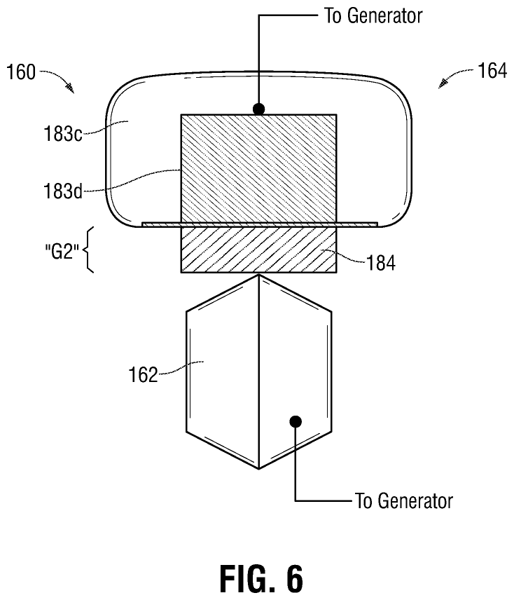

Referring to FIGS. 6-8, as an alternative to the entirety of structural body 182 of jaw member 164 being connected to generator 200 (FIG. 1), the structural body may be formed from or embedded at least partially in an insulative material, e.g., an overmolded plastic, With reference to FIG. 6, in some of such configurations, distal cap portion 183c of structural body 182 may be at least partially insulative and include a distal cap electrode 183d disposed thereon and/or therein. Distal cap electrode 183d cooperates with blade 162 to define a bipolar configuration, e.g., to act as a bipolar probe with end effector assembly 160 in the approximated position, as detailed above. Gap distance “G2” is defined between distal cap electrode 183d and blade 162.

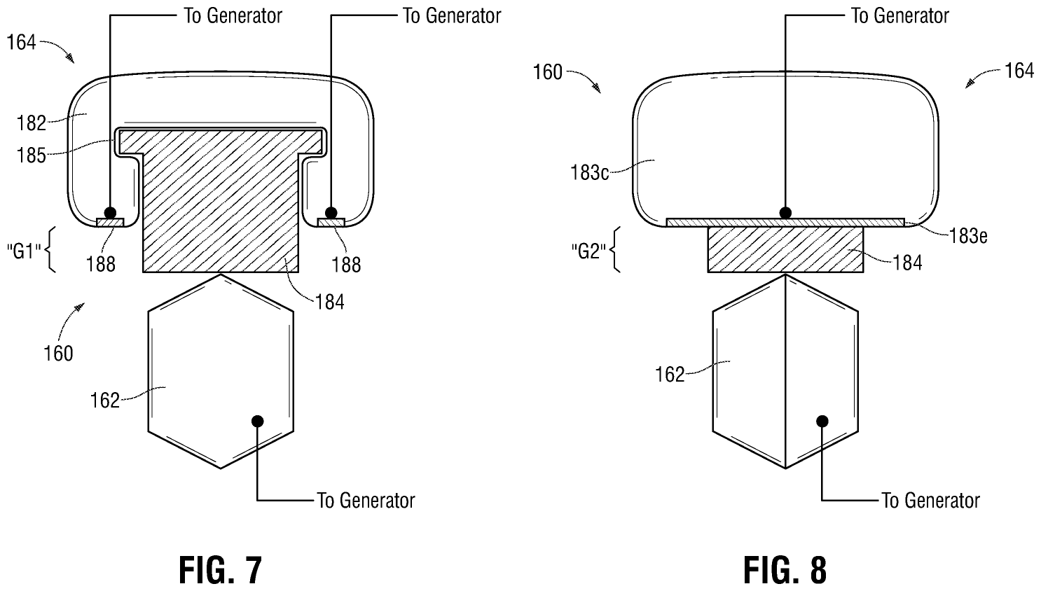

Turning to FIG. 7, in an additional or alternative aspect, the body portion of structural body 182 may be at least partially insulative and may include electrically conductive surfaces 188, e.g., in the form of plates, disposed on or captured by overmolded plastic to define electrodes on either side of jaw liner 184 on the blade facing side of the body portion of structural body 182. The electrically conductive surfaces 188 are connected to generator 200 (FIG. 1) and may be energized for use in bipolar and/or monopolar configurations, e.g., energized to the same potential as one another and/or blade 162 and/or different potentials as one another and/or blade 162. In particular, electrically conductive surfaces 188 enable end effector assembly 160 to function as a clamping tissue sealer. Gap distance “G1” is defined between electrically conductive surfaces 188 and blade 162. In aspects, electrically conductive surfaces 188 are electrically connected to or electrically isolated from distal cap electrode 183d (in configurations where both are provided). In electrically isolated configurations, electrically conductive surfaces 188 and distal cap electrode 183d may be independently activated.

FIG. 8 illustrates another configuration wherein, rather than a separate distal cap electrode 183d (FIG. 6) and electrically conductive surfaces 188 (FIG. 7), electrically conductive surfaces 188 (FIG. 7) are interconnected by a distal bridge electrode 183e that extends about the partially electrically insulative distal cap portion 183c of structural body 182. Referring also to FIG. 7, in such configurations, electrically conductive surfaces 188 (FIG. 7) and distal bridge electrode 183e cooperate to define a generally U-shaped configuration and are electrically coupled to one another. In such configurations, electrically conductive surfaces 188 enable end effector assembly 160 to function as a clamping tissue sealer while distal bridge electrode 183e enables end effector assembly 160 to function as a bipolar probe.

With general reference to FIGS. 1-8, as noted above, end effector assembly 160 is configured for use in an ultrasonic mode and/or one or more electrosurgical modes; the modes may operate consecutively, overlapping, alternatingly, simultaneous, and/or in any other suitable manner. Further, end effector assembly 160 may function as a clamping tissue sealer (and divider) in an ultrasonic mode, an electrosurgical mode, or a combined mode. In bipolar electrosurgical or combined clamping tissue sealer (and divider) modes, the gap distance “G1” between blade 162 and the corresponding electrode portion(s) of jaw member 164 facilitates RF tissue treatment, e.g., sealing. End effector assembly 160 may also function as a surgical probe in an ultrasonic mode, an electrosurgical mode, or a combined mode. In bipolar RF surgical probe modes, blade 162 and the corresponding electrode of distal cap portion 183c define gap distance “G2” therebetween to facilitate tissue treatment, e.g., spot coagulation. Other modes including the use of bipolar or monopolar electrosurgical energy are also contemplated.

With respect to ultrasonic modes, whether independent or combined with electrosurgical energy application, an ultrasonic drive signal is provided from surgical generator 200 to ultrasonic transducer 140 to generate ultrasonic energy that is transmitted from ultrasonic transducer 140 along waveguide 154 to blade 162 to thereby vibrate blade 162 for treating tissue in contact with or adjacent to blade 162. More specifically, ultrasonic energy may be supplied to blade 162 to treat, e.g., seal and/or transect (divide), tissue clamped between blade 162 and jaw liner 184 of jaw member 164; ultrasonic energy may be supplied to blade 162 to treat, e.g., transect (divide), perform an otomy, backscore, etc., tissue in contact with or adjacent to blade 162 (with jaw member 164 disposed in the spaced apart or approximated position), statically or dynamically; and/or ultrasonic energy may be supplied to blade 162 to treat, e.g., plunge, spot coagulate, etc., tissue utilizing the distal end of blade 162. The ultrasonic mode may include one or more energy level settings such as, for example, a first, e.g., LOW, setting and a second, e.g., HIGH, setting. The first and second energy level settings may correspond to different vibration velocities of blade 162.

The above-detailed aspects and features of the present disclosure enable the flexibility to alternate between modes depending upon a particular purpose and without the need to swap instruments. For example, monopolar RF energy can be used for dissection and spot coagulation, bipolar RF energy can be used for tissue sealing or assisting with tissue sealing together with ultrasonic energy, and ultrasonic energy can be used for fast bulk dissection, for tissue sealing, and/or to facilitate tissue sealing together with bipolar RF energy.

While several aspects of the disclosure have been detailed above and are shown in the drawings, it is not intended that the disclosure be limited thereto, as it is intended that the disclosure be as broad in scope as the art will allow and that the specification be read likewise. Therefore, the above description and accompanying drawings should not be construed as limiting, but merely as exemplifications of particular aspects. Those skilled in the art will envision other modifications within the scope and spirit of the claims appended hereto.

What is claimed is:

1. An end effector assembly of a surgical instrument, comprising:
an ultrasonic blade adapted to receive ultrasonic energy to vibrate the ultrasonic blade and adapted to connect to a source of electrosurgical energy, the ultrasonic blade defining a distal face; and
a jaw member movable relative to the ultrasonic blade from a spaced-apart position to an approximated position for clamping tissue therebetween, the jaw member including:
a structural body having a body portion and a distal cap portion, at least a portion of the distal cap portion of the structural body defining an electrode adapted to connect to the source of electrosurgical energy at a potential different from a potential of the ultrasonic blade; and
a jaw liner engaged within the body portion of the structural body and extending therefrom towards the ultrasonic blade such that, in the approximated position, the jaw liner contacts the ultrasonic blade to define a gap distance between the electrode and the distal face of the ultrasonic blade to facilitate bipolar electrosurgical tissue treatment upon conduction of bipolar energy between the electrode and the distal face of the ultrasonic blade and through tissue in contact therewith.

2. The end effector assembly according to claim 1, wherein the entire distal cap portion defines the electrode.

3. The end effector assembly according to claim 1, wherein a portion of the distal cap portion is electrically insulative and wherein a distal cap electrode disposed on or within the electrically insulative portion defines the electrode.

4. The end effector assembly according to claim 1, wherein a distal-most extent of the electrode and a distal-most extent of the distal face of the ultrasonic blade are substantially aligned with one another in the approximated position.

5. The end effector assembly according to claim 1, wherein the distal cap portion encloses a distal face of the jaw liner.

6. The end effector assembly according to claim 1, wherein the jaw liner is formed from an electrically-insulative material.

7. The end effector assembly according to claim 1, wherein the jaw liner is formed from a more-compliant material and the structural body is formed from a more-rigid material.

8. The end effector assembly according to claim 1, wherein the body portion of the structural body includes first and second electrode surfaces extending along opposing sides of the jaw liner, the first and second electrode surfaces adapted to connect to the source of electrosurgical energy at a potential different from the potential of the ultrasonic blade to enable conduction of bipolar energy between the first and second electrode surfaces and the ultrasonic blade and through tissue clamped therebetween in the approximated position.

9. The end effector assembly according to claim 8, wherein the electrode and the first and second electrode surfaces are independently energizable.

10. The end effector assembly according to claim 8, wherein the electrode and the first and second electrode surfaces are electrically coupled to one another.

11. The end effector assembly according to claim 8, wherein, in the approximated position, the ultrasonic blade and the first and second electrode surfaces define a second gap distance therebetween.

12. The end effector assembly according to claim 11, wherein the gap distance and the second gap distance are different from one another.
